(19) United States
(12) Patent Application Publication
(10) Pub. No.: US 2026/0015602 A1
(43) Pub. Date: Jan. 15, 2026

(54) METHOD FOR ENHANCING ANTI-TUMOR FUNCTION OF T LYMPHOCYTE AND USE THEREOF

(71) Applicants: PEKING UNIVERSITY SCHOOL OF STOMATOLOGY, Beijing (CN); BEIJING PIEZO-DENT MEDICAL TECHNOLOGY CO., LTD, Beijing (CN)

(72) Inventors: Xuehui ZHANG, Beijing City (CN); Xuliang DENG, Beijing City (CN); Dan LV, Beijing City (CN); Jia SONG, Beijing City (CN)

(21) Appl. No.: 19/115,517

(22) PCT Filed: Dec. 30, 2022
(86) PCT No.: PCT/CN2022/143783

§ 371 Date: Mar. 26, 2025

(30) Foreign Application Priority Data
Sep. 27, 2022 (CN) 202211179978.X

(51) Int. Cl. C12N 13/00; A61K 40/11; A61K 40/31; C12N 5/0783

(52) U.S. Cl. CPC C12N 13/00; A61K 40/11; A61K 40/31; C12N 5/0636; C12N 2529/00; C12N 2533/30

(57) ABSTRACT

Disclosed are a method for enhancing the anti-tumor function of a T lymphocyte and use. The method comprises the step of culturing a T lymphocyte under a physical electrical stimulation condition, wherein the T lymphocyte comprises an initial T cell or a modified T lymphocyte. The method does not involve complex traditional gene editing means with lengthy screening processes, can rapidly and effectively enhance the anti-tumor function of the T lymphocyte, and can be used for promoting CAR-T cell therapy.

### TECHNICAL FIELD

The present disclosure relates to the field of biotechnology, and in particular, to a method for enhancing the anti-tumor function of a T lymphocyte using a physical electrical stimulation, the T lymphocyte obtained by the method, and use thereof.

### BACKGROUND

With the dramatic decrease in mortality from infectious diseases, cancers, posing a great threat to human health, have become the second leading cause of death. Moreover, the incidence of cancer is on the rise year by year and younger cases have become a trend. Conventional methods for treating tumors include surgery, radiotherapy, chemotherapy, intervention, and the like. Although these methods may achieve a quick effect on reducing tumor burden, they have the disadvantages of insufficient therapeutic effects, greater adverse effects, and the like. With the progress of biotechnology and studies, a major breakthrough in tumor immunotherapy has been made in tumor treatment. Tumor immunotherapies include immune checkpoint inhibitors, adoptive cell therapies, tumor vaccines, and the like. The chimeric antigen receptor (CAR)-T cell therapy is currently the most intensely focused adoptive cell therapy, which shows great potential in treating hematological tumors, especially relapsed/refractory acute or chronic leukemia. The general process for CAR-T cell therapy comprises: isolating T lymphocytes from a tumor patient, artificially synthesizing a CAR, transducing the T lymphocytes with the designed CAR via a virus vector, screening and expanding in vitro, and transfusing the CAR-T cells back into the patient. The expressed antibody on the cell surface specifically recognizes the tumor cells and activates downstream signal pathways, thereby achieving the proliferation and activation of the CAR-T cells and specific killing of tumor cells. Compared to conventional T cell adoptive therapies, CAR-T cells can directly kill tumor cells without the participation of MHC molecules.

Although CAR-T cell therapy has a significant therapeutic effect on hematological tumors, its efficacy is subject to the short-term cell viability in the recipient body after transfusion, the poor killing potency, the insufficient infiltration in the tumor microenvironment, and the like. Therefore, maintaining the in vivo viability of CAR-T cells, optimizing the CAR-T structure, and ameliorating the tumor microenvironment are pivotal to improving the accuracy of T cells in targeting tumors and enhancing the anti-tumor effect. With the progress of research, CAR-T modified by immune regulatory molecules such as cytokines, cytokine receptors, chemokines, chemokine receptors, and chimeric activating receptors has been gradually developed to enhance the persistence and killing potency of T lymphocytes. The method for modifying T lymphocytes by gene editing has the defects of longer time consumption, low efficiency, and the like, and the random insertion of gene fragments also entails bio-safety problems. The strategy of ameliorating the tumor microenvironment is subject to individual differences in tumor patients and has no general significance.

The information provided in BACKGROUND is illustrative only of the general background of the present disclosure and should not be construed as an acknowledgment or in any way imply that this information forms the prior art, which is well known to those of ordinary skill in the art.

### SUMMARY

In order to solve at least one of the technical problems in the prior art, in particular, the technical problems of time-consuming, low efficiency, and longer screening processes in enhancing CAR-T cell therapies by means of gene editing, the present disclosure provides a method for enhancing the anti-tumor function of T lymphocytes. Specifically, the present disclosure comprises the following contents. In a first aspect of the present disclosure, provided is a method for enhancing T lymphocyte function comprising: culturing a T lymphocyte in a physical electrical stimulation condition.

In some embodiments of the method for enhancing T lymphocyte function according to the present disclosure, the physical electrical stimulation is generated by a charged matrix, a conductive matrix, a direct current, an alternating current, an electrical pulse, a magnetoelectric material or ferroelectric material. In this sense, the method in the present disclosure may also be referred to as a method for enhancing T lymphocyte function on the basis of the electrical characteristics of a material. Preferably, the ferroelectric material has a piezoelectric constant of 1-40 pC/N.

In some embodiments of the method for enhancing T lymphocyte function according to the present disclosure, the ferroelectric material comprises an organic ferroelectric polymer selected from the group consisting of a polyester, polyvinylidene fluoride, poly(vinylidene fluoride-trifluoroethylene), poly(vinylidene fluoride-hexafluoropropylene), poly(vinylidene fluoride-tetrafluoroethylene), polymethyl methacrylate, and polydimethylsiloxane, or mixtures thereof.

In some embodiments of the method for enhancing T lymphocyte function according to the present disclosure, the ferroelectric material comprises an inorganic ferroelectric particle selected from the group consisting of barium titanate, barium strontium titanate, lithium niobate, potassium sodium niobate, and hydroxyapatite, or mixtures thereof.

In some embodiments of the method for enhancing T lymphocyte function according to the present disclosure, the surface of the inorganic ferroelectric particle is coated with a dopamine layer.

In some embodiments of the method for enhancing T lymphocyte function according to the present disclosure, the method further comprises: activating and expanding the T lymphocyte.

In some embodiments of the method for enhancing T lymphocyte function according to the present disclosure, the enhanced T lymphocyte function comprises at least one selected from the following:
a. longer survival;
b. enhanced proliferation;
c. increased anti-tumor activity;
d. increased CD25 and/or DCFDA expression.

In a second aspect of the present disclosure, provided is a function-enhanced lymphocyte prepared by the method according to the first aspect of the present disclosure.

In some embodiments of the function-enhanced lymphocyte according to the present disclosure, the T lymphocyte includes an effector T cell, a cytotoxic T cell, a naive T cell, and a modified T lymphocyte, wherein the modified T lymphocyte includes a CAR-T cell.

In a third aspect of the present disclosure, provided is a pharmaceutical composition comprising the function-enhanced T lymphocyte according to the second aspect of the present disclosure.

The method in the present disclosure does not need complex conventional gene editing means with longer screening processes and can rapidly and effectively enhance the anti-tumor function of T lymphocytes. Therefore, the present disclosure may be used for enhancing CAR-T cell therapies.

### BRIEF DESCRIPTION OF THE DRAWINGS

FIG. 1 illustrates the exemplary experimental procedures for enhancing the anti-tumor function of T lymphocytes.

FIG. 2 illustrates the piezoelectric constant d₃₃ of a ferroelectric nanocomposite film material.

FIG. 3 illustrates the effect of the ferroelectric nanocomposite film material and the T lymphocytes in Example 1 and Comparative Example on the tumor volume.

FIG. 4 illustrates the flow cytometry results for the surface marker expression in OT-1 mouse CD8⁺ T lymphocytes, wherein, MFI denotes mean fluorescence intensity, and DCFDA denotes the cellular reactive oxygen species probe.

FIG. 5 illustrates the assay results of the effect of the ferroelectric nanocomposite film material with different electrical charges on the in vitro killing potency of T cells.

### DETAILED DESCRIPTION

Various exemplary embodiments of the present disclosure are described in detail below. The detailed description should not be construed as limitations on the present disclosure but as a more detailed description of certain aspects, features, and embodiments of the present disclosure.

It will be appreciated that the terms used herein are for the purpose of illustrating particular embodiments only, rather than limiting the present disclosure. In addition, for the numerical ranges in the present disclosure, it will be appreciated that the upper and lower limits of the ranges are specifically disclosed, as well as every intervening value between them. Every smaller range between any stated value or intervening value in a stated range and any other stated or intervening value in that stated range is encompassed within the present disclosure. The upper and lower limits of these smaller ranges may independently be included or excluded in the range.

Unless defined otherwise, all technical and scientific terms used herein have the same meaning as commonly understood by those of ordinary skill in the art to which the present disclosure pertains. Although only preferred methods and materials are described herein, any methods and materials similar or equivalent to those described herein can be used in the practice or testing of the present disclosure. All documents described herein are incorporated by reference to disclose and describe the methods and/or materials associated with the documents. In case of conflict with any incorporated document, the specification shall prevail. In one aspect of the present disclosure, provided is a method for enhancing T lymphocyte function, especially a method for enhancing T lymphocyte function in vitro, comprising: culturing a T lymphocyte under a physical electrical stimulation. In some embodiments, the physical electrical stimulation is provided by an external electric field. The external electric field is configured so as to allowing the surface in contact with T lymphocytes is negatively charged.

In some embodiments, the surface is negatively charged by using a ferroelectric material to apply the physical electrical stimulation. The ferroelectric material has a piezoelectric constant of 1-40 pC/N, preferably 5-30 pC/N, more preferably 6-35 pC/N, further more preferably 8-30 pC/N, such as 10 pC/N, 15 pC/N, 18 pC/N, and 20 pC/N.

In some embodiments, the ferroelectric material comprises an organic ferroelectric polymer, which selected from the group consisting of a polyester, polyvinylidene fluoride, poly(vinylidene fluoride-trifluoroethylene), poly(vinylidene fluoride-hexafluoropropylene), poly(vinylidene fluoride-tetrafluoroethylene), polymethyl methacrylate, and polydimethylsiloxane, or mixtures thereof. As used herein, polyvinylidene fluoride is also sometimes referred to as polyvinylidene difluoride (also abbreviated as PVDF), polymethyl methacrylate as PMMA, and polydimethylsiloxane as PDMS. In the present disclosure, examples of the polyester include, but are not limited to, poly(L-lactide) (also abbreviated as PLLA), poly(lactic-co-glycolic acid) (also abbreviated as PLGA), and polycaprolactone (also abbreviated as PCL).

The molecular weight of the ferroelectric polymer is not particularly specified in the present disclosure as long as the objective of the present disclosure can be achieved. For example, the weight average molecular weight of the above PVDF, poly(vinylidene fluoride-trifluoroethylene), and poly(vinylidene fluoride-hexafluoropropylene) may be respectively 150,000-300,000, 100,000-200,000, and 700,000-900,000.

In the present disclosure, the ferroelectric material comprises an inorganic ferroelectric particle which selected from the group consisting of barium titanate, barium strontium titanate, lithium niobate, potassium sodium niobate, and hydroxyapatite, or mixtures thereof. Preferably, the inorganic ferroelectric particle has a particle size of 0.1-500 nm, more preferably 10-50 nm. In exemplary embodiments, the ferroelectric material of the present disclosure comprises both the ferroelectric polymer and the ferroelectric particle in a ratio by mass of 1:1-1:10, preferably 1:1-1:5, and more preferably 1:1-1:4.

In the present disclosure, the inorganic ferroelectric particle is further modified by coating with a dopamine layer to provide excellent biocompatibility and ferroelectric properties. In the present disclosure, by coating the surface of a ceramic nanoparticle with dopamine, a transition bonding interlayer is introduced between the ceramic nanoparticle and the ferroelectric polymer, thus effectively improving the interface between the ceramic nanoparticle and the ferroelectric polymer, reducing the defects caused by poor interface compatibility, achieving uniform dispersion of the ceramic nanoparticle in the ferroelectric polymer, and improving the ferroelectric properties of the material.

In the present disclosure, the thickness of the dopamine layer is not particularly specified, which may be 1-10 nm, such as 1, 2, 3, 4, 5, 6, 7, 8, 9 or 10 nm, or a thickness of any value therebetween.

In the present disclosure, the ratio by mass of dopamine to the ferroelectric particle is 1:1-10:1, preferably 2:1-8:1, and more preferably 3:1-7:1.

In some embodiments, a substrate specific for culturing a T lymphocyte and therefore enhancing its anti-tumor function is prepared from the ferroelectric material of the present disclosure. Preferably, the culture-specific substrate comprises a positively charged first surface and a negatively charged second surface. The thickness of the substrate formed by the ferroelectric material is not particularly specified, which may be, preferably 10-80 μm, more preferably 15-50 μm, and further more preferably 20-40 μm. Although both the first surface and the second surface have good ferroelectric properties, only the negatively charged second surface, but not the positively charged first surface, can enhance the anti-tumor function of T lymphocytes in an assay of in-vitro T cell killing potency. The second surface further inhibits the tumor size and increases the quantities of biomarkers related to the viability of T lymphocytes in a charge intensity-and dose-dependent trend.

In the present disclosure, the method for film preparation is not particularly specified and may be a method known in the art. Examples of the method include, but are not limited to, spin coating, casting, screen printing, dip coating, inkjet printing, spray pyrolysis, and the like.

In the present disclosure, the ferroelectric material is obtained by annealing and/or polarization treatment, and the order of annealing and polarization is not particularly specified.

In some embodiments, the film material is annealed at 50-150° C. for 0.5-3 h and cooled to room temperature. The film material after the treatments is then subjected to a corona polarization treatment to give the ferroelectric material, wherein the voltage of the corona polarization treatment is 10-50 kV, the polarization temperature is 25-50° C., and the duration is 10-60 min. Preferably, the annealing treatment temperature is 90-140° C. and the duration is 2-3 h; the voltage of the corona polarization treatment is 20-50 kV, the temperature is 25-40° C., and the duration is 10-40 min. For example, the annealing treatment temperature may be 90° C., 100° C., 110° C., 120° C., 130° C., 140° C., or a range delimited by any values therebetween, and the duration of the annealing treatment may be 0.5 h, 1 h, 1.5 h, 2.0 h, 2.5 h, 3 h, or a range delimited by any values therebetween. For example, the voltage of the corona polarization treatment may be 10 kV, 20 kV, 30 kV, 40 kV, 50 kV, or a range delimited by any values therebetween, the temperature of the corona polarization treatment may be 25° C., 30° C., 35° C., 40° C., 45° C., 50° C., or a range delimited by any values therebetween, and the duration of the corona polarization treatment may be 10 min, 20 min, 30 min, 40 min, 50 min, 60 min, or a range delimited by any values therebetween.

In some embodiments, the ferroelectric material is obtained by subjecting the film material to a corona polarization treatment and then an annealing treatment at 50-150° C. for 0.5-3 h, wherein the voltage of the corona polarization treatment is 10-50 kV, the temperature is 25-50° C., and the duration is 10-60 min; preferably, the voltage of the corona polarization treatment is 20-50 kV, the temperature is 25-40° C., and the duration is 10-40 min; the annealing treatment temperature is 90-140° C. and the duration is 0.5-2 h. For example, the voltage of the corona polarization treatment may be 10 kV, 20 kV, 30 kV, 40 kV, 50 kV, or a range delimited by any values therebetween, the temperature of the corona polarization treatment may be 25° C., 30° C., 35° C., 40° C., 45° C., 50° C., or a range delimited by any values therebetween, and the duration of the corona polarization treatment may be 10 min, 20 min, 30 min, 40 min, 50 min, 60 min, or a range delimited by any values therebetween. For example, the annealing treatment temperature may be 90° C., 100° C., 110° C., 120° C., 130° C., 140° C., or a range delimited by any values therebetween, and the duration of the annealing treatment may be 0.5 h, 1 h, 1.5 h, 2.0 h, 2.5 h, 3 h, or a range delimited by any values therebetween.

The corona polarization treatment can endow the surface of the film material with more polarization charges and thus improve its ferroelectric properties. A corona polarization treatment at an insufficient voltage (e.g., below 10 kV), at a lower temperature (e.g., below 25° C.), or in a shorter duration (e.g., below 10 min), may be inadequate and directly affect the improvement of ferroelectric properties of the film material. A corona polarization treatment at an excessive voltage (e.g., above 50 kV), at a higher temperature (e.g., above 50° C.), or in a longer duration (e.g., above 60 min), may bring safety concerns to the actual operation and affect the mechanical properties of the film material.

In the present disclosure, the T lymphocyte includes, but is not limited to, at least one of an effector T cell, a cytotoxic T cell, a naive T cell, and a modified T lymphocyte, or mixtures thereof, wherein the modified T lymphocyte includes a CAR-T cell. In the present disclosure, a CD8⁺ T lymphocyte is obtained by sorting naive T lymphocytes provided by a donor, and then activated by OVA peptide and expanded via cytokine induction. It will be appreciated that although in some embodiments, naive T lymphocytes, CD8⁺ T lymphocytes, the OVA peptide activation, and the cytokine-induced expansion are used, these, however, are not intended to limit the particular types of the T lymphocyte or the method for T lymphocyte preparation, and one skilled in the art can select an appropriate T lymphocyte, donor, or method for activation as desired to obtain the T lymphocyte capable of inhibiting tumors.

In the present disclosure, the function-enhanced T lymphocyte includes a modified T lymphocyte. The term “modified T lymphocyte” refers to a T lymphocyte that is transduced, using gene modification technology, with genetic materials encoding a specific antigen recognition domain and a T cell activation signal, such that the T cell can be activated by directly binding to a specific antigen on the surface of a tumor cell and kill the tumor cell by releasing killing proteins, killing factors, and the like. The T cell may further recruit endogenous immune cells by releasing cytokines to kill the tumor cell in the human body, thereby achieving the purpose of treating the tumor. Furthermore, a specific long-term anti-tumor mechanism is built via the forming of an immunological memory T cell. Preferably, the modified T lymphocyte is a CAR-T cell.

In exemplary embodiments, the method for preparing the enhanced T lymphocytes of the present disclosure comprises at least the following steps:
1) preparing a ferroelectric nanocomposite film material;
2) acquiring a naive T lymphocyte from a donor;
3) culturing the T lymphocyte on the surface of the ferroelectric nanocomposite film material; and
4) activating and expanding the T lymphocyte.

Dopamine or a salt thereof and the inorganic ferroelectric particles are dispersed in water in a certain ratio by ultrasonication, and the dispersion is stirred and dried in vacuum at 40-70° C. to give the modified ferroelectric particles. Then a certain amount of organic ferroelectric polymer is mixed with an organic solvent, and the mixture is stirred; the modified ferroelectric particles are dispersed in the organic solvent by ultrasonication to give a dispersion. The dispersion is dropwise added to the organic solution containing the organic ferroelectric polymer, and the mixture is stirred to give a uniform liquid mixture. A nanocomposite film with a specific thickness is obtained by the casting method, and the organic solvent residue in the nanocomposite film is volatilized after drying.

In the preparation method of the present disclosure, the organic ferroelectric polymer is present in the organic solvent at a concentration of 0.1-2 g/mL, preferably 0.1-1.5 g/mL. The modified ferroelectric particle is present in the organic solvent at a concentration of 10-100 g/L, preferably 40-60 g/L. The organic solvent is not particularly specified, including, but not limited to, an alcohol solution, N,N-dimethylformamide, dimethylsulfoxide, and the like.

In the preparation method of the present disclosure, the OVA peptide for inducing the T lymphocyte activation is at a concentration of 0.1-4 μg/mL, preferably 0.15-3 μg/mL.

In the preparation method of the present disclosure, the T lymphocyte expansion is induced by a cytokine; preferably, the cytokine is IL-2 at a concentration of 0.1-4 ng/mL, preferably 0.15-3 ng/mL.

The anti-tumor function of the T lymphocyte can be determined by the tumor size and tumor growth, or the expression level of T lymphocyte surface markers and the survival rate of tumor cells. The assay of markers may be methods known in the art, including, but not limited to, for example, flow cytometry, immunoblotting, and the like. In some embodiments, the marker includes the survival-related index CD25 and the reactive oxygen species metabolism-related index DCFDA.

It will be appreciated by those skilled in the art that other procedures or operations, such as further optimization and/or improvement of the method of the present disclosure, may be included before, after, or between any of steps (1)-(6) described above, as long as the objective of the present disclosure can be achieved.

### Example

### (1) Preparation of Ferroelectric Nanocomposite Film Material

0.3 g of dopamine hydrochloride powder was mixed with 180 mL of deionized water. After uniformly stirring, 6 g of barium titanate nanoparticles were added and the mixture was ultrasonicated for 30 min for uniform dispersion. The resultant mixture was stirred for 10-12 h. The solution was left to stand until the phases were separated. The supernatant was discarded, and the residue was repeatedly washed using absolute ethanol and deionized water until the supernatant was clear and no noticeable turbidity was observed. The dopamine hydrochloride-modified barium titanate nanoparticles were baked in a thermostatic vacuum drying oven at 55° C. until the particles were completely dried.

5 g of P(VDF-TrFE) powder was added to 35 mL of N,N-dimethylformamide (DMF), and the mixture was stirred for 3 h. 0.89 g of dopamine hydrochloride-modified barium titanate nanoparticles were added to 15 mL of DMF. The mixture was stirred for 30 min and ultrasonicated for 30 min for 3 repeated cycles to adequately homogenize the material. The dispersion of dopamine hydrochloride-modified barium titanate nanoparticles after ultrasonication was dropwise added to a P(VDF-TrFE) solution, and the mixture was stirred for 12 h to give a uniform and stable BaTiO₃/P(VDF-TrFE) mixed solution. A BaTiO₃/P(VDF-TrFE) ferroelectric nanocomposite film with a thickness of 30 μm was applied to a quartz glass by the casting method and was dried on a heating platform at a constant temperature of 55° C., until the solvent was completely volatilized.

In this experiment, the annealing treatment was selected to promote the crystallization and β phase formation in PVDF, so as to further increase the content of β phase in the BaTiO₃ nanoparticle-doped P(VDF-TrFE) ferroelectric film. The materials in the NC and LC groups were annealed on a heating platform at 55° C. for 30 min. Then the materials in the MC group were annealed on the heating platform at 90° C. for 30 min, while the materials in the HC group were annealed on the heating platform at 120° C. for 30 min. After the films were cooled to room temperature on the heating platform, the films were gently separated from the quartz plate.

According to the corona polarization method, the prepared BaTiO₃/P(VDF-TrFE) ferroelectric nanocomposite film material was placed on the metal base plate of a high-voltage direct-current polarizer and processed at a loading voltage of 21 kV (21.5 kV for HC group) for 30 min of polarization at room temperature.

### (2) Acquisition of Naive T Lymphocytes From Donors

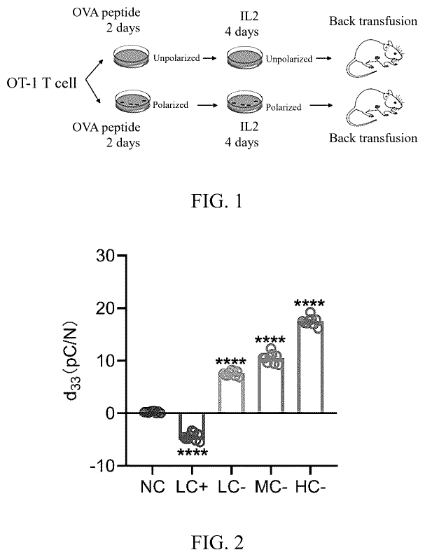

As shown in FIG. 1, OT-1 mice were sacrificed by cervical dislocation, fixed on a wax plate with needles, and disinfected with 75% alcohol. The abdominal skin of the mouse was clipped using ophthalmic forceps, and a small incision along the ventral midline was made using ophthalmic scissors. Blunt separation of the peritoneum and skin was conducted using ophthalmic scissors and the skin was cut along the ventral midline to expose the entire peritoneum. The peritoneum was clipped using ophthalmic forceps and cut along the ventral midline with ophthalmic scissors to expose the spleen tissue. Blunt separation of the spleen and the surrounding tissues was performed using ophthalmic forceps, and the spleen was placed in a 10 cm petri dish containing 13 mL of FACS buffer pre-cooled at 4° C.

A 200-mesh screen was placed in a 10 cm petri dish and immersed in FACS buffer. The spleen tissue was transferred to the 200-mesh screen using ophthalmic forceps and minced with ophthalmic scissors. The minced spleen tissue was ground in the same direction on the 200 mesh screen using a grinding rod until no noticeable tissue mass was observed. In the grinding, the 200-mesh screen should neither contact the petri dish bottom nor detach from the FACS buffer to avoid the death of the spleen cells. The FACS buffer was removed from the petri dish by pipetting, and the 200-mesh screen was repeatedly washed to collect the remaining cells. All FACS buffer in the petri dish was transferred to a 15 mL centrifuge tube by pipetting and centrifuged for 5 min at 1600 rpm and 4° C. using a horizontal rotor centrifuge, and the supernatant was discarded. The pelleted cells were treated with 1 mL of ACK lysis buffer for 5 min at room temperature. The lysis was stopped by adding 10 mL of FACS buffer. After centrifugation, the pelleted cells were resuspended using 1 mL of FACS buffer, and CD8⁺ T lymphocytes were sorted using magnetic beads for subsequent experiments.

(3) Culture of T lymphocytes on Surface of Ferroelectric Nanocomposite Film Material

The CD8⁺ T lymphocytes obtained from the previous step were cultured on unpolarized (UP) and polarized (P) BaTiO₃/P(VDF-TrFE) ferroelectric nanocomposite films at a concentration of 2×10⁶ cells/mL.

### (4) Activation and Expansion of T Lymphocytes

OT-1 mouse CD8⁺ T lymphocytes were activated for 2 d by induction with 2 μg/mL of OVA peptide, and the dead cells from the activation were removed using a mouse lymphocyte separation medium and centrifugation without brake.

IL-2 was added at 2 ng/ml to induce the expansion of mouse CD8⁺ T lymphocytes for 4 days. The cells were collected and counted daily. The cell density was adjusted to 2×10⁶ cells/mL, and the dead cells from the expansion were removed using the mouse lymphocyte separation medium and centrifugation without brake.

### (5) Transfusion of T Lymphocytes Back to Tumor-Bearing Recipients

NOD-SCID mice were subcutaneously grafted at the left flank with 3×10⁶ LLC tumor cells overexpressing OVA. On day 7 of the tumor cell grafting, 5×10⁵ OT-1 mouse CD8⁺ T lymphocytes after in vitro activation and expansion were intravenously injected via the tail vein of the tumor-bearing mouse.

### (6) Detection of Anti-Tumor Function of T Lymphocytes

The subcutaneous tumor size was continuously observed and measured using length×width² of the tumor in the mouse as the tumor volume. The expression level of the OT-1 mouse CD8⁺ T lymphocyte surface markers was determined by flow cytometry.

### (7) Detection of Effect of Ferroelectric Nanocomposite Film Material With Different Electrical Charges on in Vitro Killing Potency of T Cells

OT-1 mouse CD8⁺ T lymphocytes were acquired, plated and cultured on ferroelectric nanocomposite films with different electric charges at a concentration of 2×10⁶ cells/mL, and activated using 2 μg/mL of OVA peptide for 2 days. The activated CD8⁺ T lymphocytes were added to the tumor cells in a ratio of 1:1, 0.5:1, or 0.25:1 (T cells:tumor cells), and the survival of the tumor cells was determined after 24 h.

### Comparative Example

The BaTiO₃/P(VDF-TrFE) ferroelectric nanocomposite film was not polarized after being dried on a heating platform at a constant temperature of 55° C., with other procedures remaining identical to those in Example 1.

### Test Example

The results of the piezoelectric constant d₃₃ of the ferroelectric nanocomposite film material are shown in FIG. 2, wherein NC denotes no charges; LC+ denotes low positive charges (annealing temperature 55° C., polarization voltage 21 kV); LC− denotes low negative charges (annealing temperature 55° C., polarization voltage 21 kV); MC-denotes medium negative charges (annealing temperature 90° C., polarization voltage 21 kV); HC-denotes high negative charges (annealing temperature 120° C., polarization voltage 21.5 kV). In addition, the piezoelectric constants d₃₃ of the ferroelectric nanocomposite film material prepared in Comparative Example were determined to be −4.678±0.2525 (LC+), 7.322±0.1637 (LC−), 10.27±0.3297 (MC−), and 17.27±0.2864 (HC−), respectively; wherein, the low charge refers to 4-8 pC/N, the medium charge refers to 9-12 pC/N, and the high charge refers to 15-20 pC/N.

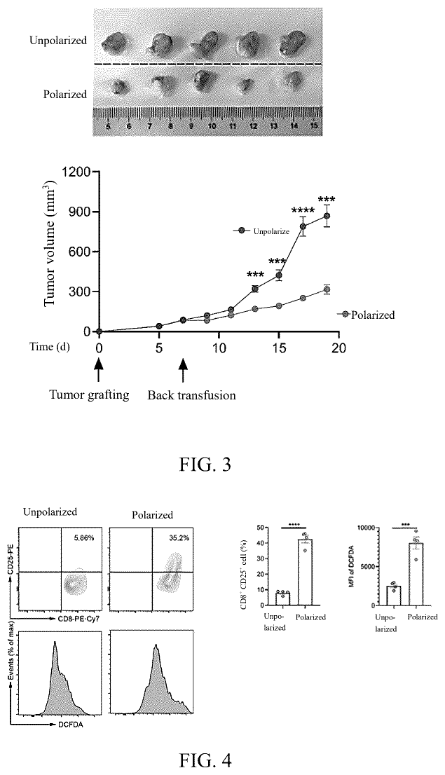

The T lymphocytes cultured on the surface of the polarized ferroelectric nanocomposite film material exhibited a stronger anti-tumor potency, which is demonstrated by a smaller tumor volume and a lower tumor growth rate (see FIG. 3) after the T lymphocytes were transfused back to the mice. The T lymphocytes cultured on the surface of the unpolarized ferroelectric nanocomposite film material, compared to the polarized counterpart, exhibited a weaker anti-tumor potency, which is demonstrated by a greater tumor volume and a higher tumor growth rate (see FIG. 3) after the T lymphocytes were transfused back to the mice. The expression level of the OT-1 mouse CD8⁺ T lymphocyte surface markers was determined by flow cytometry. The results show that the survival-related index CD25 and the reactive oxygen species metabolism-related index DCFDA of the CD8⁺ T lymphocytes cultured on the surface of the polarized ferroelectric nanocomposite film material increased to some extent. The survival-related index CD25 and the reactive oxygen species metabolism-related index DCFDA of the CD8⁺ T lymphocytes cultured on the surface of the unpolarized ferroelectric nanocomposite film material, compared to the polarized counterpart, were lower (see FIG. 4).

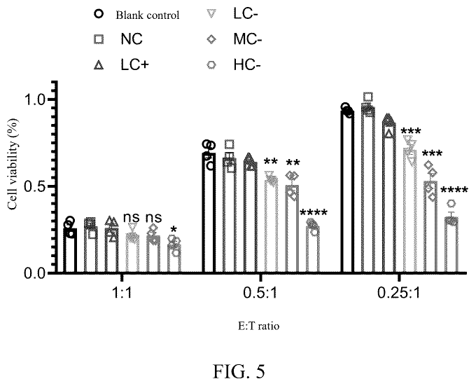

As shown in FIG. 5, in the assay of the effect of the ferroelectric nanocomposite film material on in vitro killing potency of T cells, OT-1 mouse CD8⁺ T lymphocytes were acquired, and plated and cultured on ferroelectric nanocomposite films with different electric charges. The CD8⁺ T lymphocytes were activated by induction. The activated CD8⁺ T lymphocytes were added to tumor cells in different (T cells:tumor cells) ratios. The 24 h tumor cell survival results demonstrate that the CD8⁺ T lymphocytes significantly inhibited the tumor cell survival in a charge intensity- and dose-dependent manner when being cultured on the negatively charged surface of the ferroelectric nanocomposite film. On one hand, the inhibitory effect on tumor cell survival was more significant when the (T cells:tumor cells) ratio was close to 1:1. On the other hand, the inhibitory effect on tumor cell survival was increasingly significant with the increase of the electric charge or the piezoelectric constant.

Although the present disclosure has been described with reference to exemplary embodiments, it will be appreciated that the present disclosure is not limited to the disclosed exemplary embodiments. Various modifications and variations can be made to the exemplary embodiments in the specification of the present disclosure without departing from the scope or spirit of the present disclosure. The claimed scope should be based on the broadest interpretation to encompass all modifications and equivalent structures and functionalities.

1. A method for enhancing T lymphocyte function comprising: culturing a T lymphocyte in a physical electrical stimulation condition.

2. The method according to claim 1, wherein the physical electrical stimulation is generated by a charged matrix, a conductive matrix, a direct current, an alternating current, an electrical pulse, a magnetoelectric material or a ferroelectric material, and the ferroelectric material has a piezoelectric constant of 1-40 pC/N.

3. The method according to claim 2, wherein the ferroelectric material comprises an organic ferroelectric polymer selected from the group consisting of a polyester, polyvinylidene fluoride, poly(vinylidene fluoride-trifluoroethylene), poly(vinylidene fluoride-hexafluoropropylene), poly(vinylidene fluoride-tetrafluoroethylene), polymethyl methacrylate, and polydimethylsiloxane, or mixtures thereof.

4. The method according to claim 2, wherein the ferroelectric material comprises an inorganic ferroelectric particle selected from the group consisting of barium titanate, barium strontium titanate, lithium niobate, potassium sodium niobate, and hydroxyapatite, or mixtures thereof.

5. The method according to claim 4, wherein the surface of the inorganic ferroelectric particle is coated with a polydopamine layer.

6. The method according to claim 1, wherein the enhanced function comprises at least one selected from the group consisting of the following, or mixtures thereof:
a. longer survival;
b. enhanced proliferation;
c. increased anti-tumor activity; and
d. increased CD25 and/or DCFDA expression.

7. Use of a ferroelectric material in preparing a culture substrate specific for enhancing T lymphocyte function.

8. A function-enhanced T lymphocyte prepared by the method according to claim 1.

9. The function-enhanced T lymphocyte according to claim 8, wherein the T lymphocyte is selected from the group consisting of an effector T cell, a cytotoxic T cell, a naive T cell, and a modified T lymphocyte, or mixtures thereof, wherein the modified T lymphocyte is a CAR-T cell.

10. A pharmaceutical composition comprising the function-enhanced T lymphocyte according to claim 8.
